(19) United States
(12) Patent Application Publication
(10) Pub. No.: US 2024/0407876 A1
(43) Pub. Date: Dec. 12, 2024

(54) SURGEON INPUT TOOL FOR A SURGICAL ROBOTIC SYSTEM

(71) Applicant: Asensus Surgical US, Inc., Durham, NC (US)

(72) Inventor: Randall L. Luck, Cary, NC (US)

(21) Appl. No.: 18/739,259

(22) Filed: Jun. 10, 2024

Related U.S. Application Data
(60) Provisional application No. 63/507,441, filed on Jun. 9, 2023.
(60) Provisional application No. 63/617,753, filed on Jan. 4, 2024.

(51) Int. Cl. A61B 34/00; A61B 34/20; A61B 34/35; A61B 90/00

(52) U.S. Cl. CPC A61B 34/74; A61B 34/25; A61B 34/35; A61B 2034/2055; A61B 2034/2072; A61B 2090/3945

(57) ABSTRACT

A user input tool for providing input to a robotic surgical system for use in commanding motion of the robotic manipulator has a grip positionable in contact with a palm of a user. A lever is mounted to the grip and pivotable relative to the grip around a lever pivot axis. The lever pivot axis is offset from, and parallel to, the joint pivot axis.

### RELATED APPLICATIONS

This application claims the benefit of U.S. Provisional Application No. 63/507,441, filed Jun. 9, 2024, and U.S. Provisional Application No. 63/617,753 filed Jan. 14, 2024.

### TECHNICAL FIELD OF THE INVENTION

The present invention relates generally to the field of robot-assisted surgical devices and systems, and more particularly to devices and systems for providing user input to surgical robotic systems to cause corresponding movement of surgical instruments at a surgical site.

### BACKGROUND

There are various types of surgical robotic systems on the market or under development. Some surgical robotic systems use a plurality of robotic arms. Each arm carries a surgical instrument or the camera used to capture images from within the body for display on a monitor. Typical configurations allow two or three instruments and the camera to be supported and manipulated by the system. Input to the system is generated based on input from a surgeon positioned at a master console, typically using input devices such as input handles. Motion and actuation of the surgical instruments and the camera is controlled based on the user input. The image captured by the camera is shown on a display at the surgeon console. The console may be located patient-side, within the sterile field, or outside of the sterile field.

The robotic arms/manipulators include a portion, typically at the terminal end of the arm, that is designed to support and operate a surgical device assembly. The surgical device assembly includes a surgical instrument having a shaft and a distal end effector on the shaft. The end effector is positionable within a patient. The end effector may be one of many different types that are used in surgery including, without limitation, end effectors having one or more of the following features: jaws that open and close, a section at the distal end of the shaft that bends or articulates in one or more degrees of freedom, a tip that rolls axially relative to the shaft, a shaft that rolls axially relative to the manipulator arm.

In many robotic surgical systems, particularly those using rigid shaft instruments, the surgical instruments are both robotically manipulated by the robotic manipulator arms disposed outside the patient's body, as well as electromechanically actuated within the patient's body. Robotic manipulation pivots the instrument shaft relative to the patient and may alter the insertion depth of the instrument and/or cause the instrument to roll about its longitudinal axis. Electromechanical actuation (or hydraulic/pneumatic actuation) may open and close jaws of the instrument, and/or actuate articulating or bending of the distal end of the instrument shaft, and/or roll the instrument's shaft or distal tip. Some systems may use only this latter form of instrument motion while holding the more proximal part of the instrument in a fixed position outside the body using a fixed support or inactive robotic manipulator.

Typically, a proximal housing is positioned on the proximal end of the instrument shaft. This housing functions as an adapter or interface between the surgical instrument and the robotic manipulator arm. The adapter may house passive actuation mechanisms that receive motion transferred from active actuators in the robotic manipulator or other support to drive functions of the instrument end effector. The instrument actuators for driving the motion of the end effector, which respond to user input to cause actuation of the instrument's functions, are electromechanical motors or other types of motors (e.g., hydraulic/pneumatic). They are often positioned in the terminal portion of the robotic manipulator. In some cases, they are positioned in the proximal housing of the surgical device assembly, and for other configurations some are in the proximal housing while others are in the robotic manipulator. In the latter example, some functions of the end effector might be driven using one or more motors in the terminal portion of the manipulator while other motion might be driven using motors in the proximal housing. See, for example, US 2016/0058513, Surgical System with Sterile Wrappings, in which jaw open-close functions are initiated using electromechanical actuators in the robotic manipulator, and in which rotation or swivel functions of the instrument are initiated using electromechanical actuators housed in the proximal housing of the surgical device assembly.

In some systems, some of the surgical instruments may be removably connected to their respective adapters. This facilitates post-surgery cleaning and sterilization of the instruments and adapters, by allowing them to be separated.

For surgical instruments that are actuated to carry out jaw open-close, shaft articulating or bending, or other functions, there is typically a mechanical interface between the adapter and the robotic manipulator through which motion generated by the instrument actuators within the robotic manipulator is communicated to one or more mechanical inputs of the adapter to control any degrees of freedom of the instrument and, if applicable, its jaw open-close function. This motion may be communicated through a drape positioned between the sterile adapter and the non-sterile manipulator arm. In some commercially available robotic systems, the motion is communicated using rotary connections in which rotating disks on the manipulator transfer motion to rotating disks on an instrument adapter. See, for example, the configuration shown in U.S. Pat. No. 6,491,701. In the embodiment shown in U.S. Pat. No. 9,358,682, a transverse slider pin extends laterally from one side of the case mounted to the proximal end of the instrument. It is moveable to open and close jaws of the instrument (FIG. 18 of the patent). When the instrument is mounted to the manipulator arm, the slider pin is received by a corresponding component) in the manipulator arm. When it is necessary to open/close the instrument jaws, the component is translated on a carriage by motors in the laparoscopic instrument actuator of the manipulator arm. This advances the slider pin to actuate the jaws.

In other systems, an instrument drive system positioned on a distal part of the manipulator arm has multiple linear drive outputs, as shown and described in commonly-owned US 2021169595, Compact Actuation Configuration and Expandable Instrument Receiver for Robotically Controlled Surgical Instruments. The surgical instrument's adapter housing has corresponding linear drive inputs, each of which is moveable to modify the tension on a corresponding actuation cable of the surgical instrument. When the surgical instrument is mounted to the manipulator arm, the adapter housing is positioned in the instrument drive system such that each linear drive output is positioned to drive a linear drive input of the adapter. The instrument drive system is controlled to selectively operate motors that drive the linear drive outputs in order to selectively alter the tension on the cables as need to articulate the distal end of the instrument and open/close its jaws. The instruments may be of the type described in commonly owned application US 2020/0315722, Articulating Surgical Instrument.

The instruments are exchangeable during the course of the procedure, allowing one instrument (with its corresponding adapter) to be removed from a manipulator and replaced with another. To minimize surgical procedure time and make efficient use of operating room personnel, it is essential to make the instrument exchange process as efficient as possible.

For robust, safe instrument exchange, it is important to both ensure that the user has a secure hold on the instrument and is also in a safe position relative to a moving robotic manipulator arm.

The number of degrees of freedom (DOFs) of motion for a robotically controlled instrument can vary between surgical systems and also between the different devices used for a particular system. Likewise, instruments with varying levels of complexity can be used interchangeably on a particular type of robotic system.

For example, a robotically controlled rigid-shafted instrument that moves similarly to a conventional laparoscopic instrument will be pivoted by the robotic arm relative to a fulcrum at the incision site (instrument pitch-yaw motion), axially rolled about the instrument's longitudinal axis, and translated along the longitudinal axis of the instrument (along the axis of insertion/withdrawal of the instrument relative to the incision). Other robotically controlled rigid-shafted instruments might be configured to move in a manner similar to that described in the previous paragraph, but to also have slightly more complexity, such as one or more degrees of articulation of the end effector about the instrument shaft (e.g. pitch and jaw of the instrument end effector relative to the shaft, which may be in addition to the pitch and/or yaw that results from movement of the rigid instrument shaft about a fulcrum at the incision site), giving the instrument 6 DOFs. See, for example, the instruments described in co-pending and commonly owned application US 2020/0315722, Articulating Surgical Instrument, which is incorporated by reference. Such instruments might optionally also include the ability to axially roll the instrument's tip about the shaft.

There are other types of user instrument handle motion, besides laparoscopic motion, used in surgery. Another type of instrument handle motion used in surgery is referred to as “true cartesian motion,” which differs from laparoscopic motion in that there is no inversion of the motion, so the user input handle is raised to cause the surgical robotic system to raise the instrument tip, moved left to cause movement of the tip to the left, etc. Some surgical systems may allow surgical personnel to choose whether the system will operate in a laparoscopic type of mode or in a true cartesian motion mode. Others might make use of a surgeon console that is configured so it can be selectively used use with a laparoscopic surgical system and with a true cartesian surgical system.

Conventional surgeon consoles have input devices coupled to mechanical linkages or gimbles. More recently, consoles have been proposed in which the user input handles are tracked using electromagnetic tracking, optical tracking or other forms of tracking such as inertial tracking. Applicant's co-pending and commonly-owned application U.S. Ser. No. 18/663,595, Surgeon Input System Using Event-Based Vision Sensors for a Surgical Robotic System, describes a novel form of tracking configuration using event based vision sensor for tracking user input devices at a surgeon console for use in commanding motion of a surgical instrument of a surgical robotic system. The user input system may be used to command any of the types of motion described above, and for some robotic surgical systems it may be configured to allow the user to instruct the system as to which type of motion (e.g. laparoscopic or true cartesian) is to be commanded.

This application describes a tool configuration that may be used do give user input to a surgical robotic system. It is suitable for used in both tracked handle configurations or mechanically coupled configurations, including the types described in the preceding paragraph.

### BRIEF DESCRIPTION OF THE DRAWINGS

FIG. 1 illustrates a robotic surgical system with which the disclosed surgeon input tool may be used.

FIGS. 2A and 2B are perspective views of a right-hand input tool.

FIG. 3 is a perspective view of the input tool of FIGS. 2A and 2B held by a user.

FIGS. 4A and 4B are similar to FIG. 3, but show a top view of the input tool in the open and closed position.

FIGS. 5A and 5B are similar to FIG. 3, but show a perspective view of the input tool in the open and closed position.

FIG. 6 shows the input tool of FIGS. 2A and 2B with a first exemplary tracking frame mounted to it.

FIG. 7 shows the input tool of FIGS. 2A and 2B with a second exemplary tracking frame mounted to it.

FIG. 8A is a rear elevation view of the input tool of FIG. 7.

FIG. 8B is a front perspective view of the input tool of FIG. 7.

FIG. 9 is a perspective view showing the input tool of FIG. 7 held by a user.

FIG. 10 illustrates a surgeon console at which a user is seated and using the input tool of FIG. 7 in the user's right and left hands.

### DETAILED DESCRIPTION

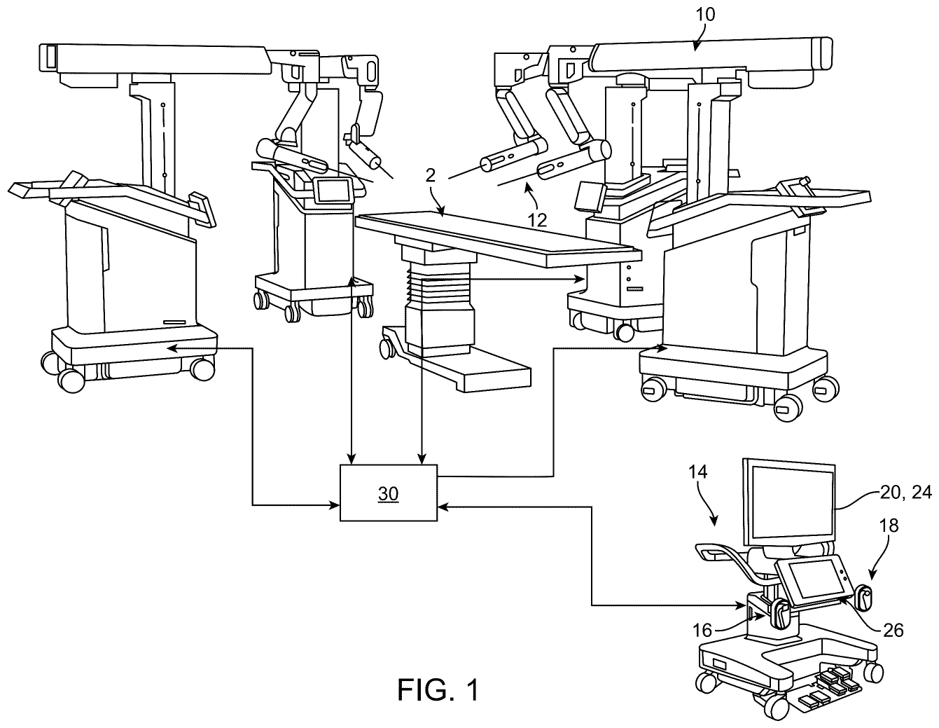

Although the concepts described in this application may be used on a variety of robotic surgical systems, the embodiments will be described with reference to a system of the type shown in FIG. 1. In the illustrated system, robotic manipulators 14 are disposed adjacent to a patient bed 2. Each manipulator 14 is configured to maneuver a surgical instrument 10 which has a distal end effector positionable in a patient body cavity. A surgeon console 12 has two input devices such as handles 17, 18. The input devices are configured to be manipulated by a user to generate signals that are used to command motion of the robotic manipulators in multiple degrees of freedom in order to maneuver the instrument end effectors within the body cavity. In use, the user selectively assigns the two handles 17, 18 to two of the robotic manipulators 14, allowing surgeon control of two of the surgical instruments 10 at any given time. To control a third one of the instruments disposed at the working site, one of the two handles 17, 18 may be operatively disengaged from one of the initial two instruments and then operatively paired with the third instrument, or another form of input may control the third instrument as described in the next paragraph. FIG. 1 shows four robotic manipulators, although in other configurations, the number of manipulators may differ.

The surgical system allows the operating room staff to remove and replace the surgical instruments 10 carried by the robotic manipulator, based on the surgical need. When an instrument exchange is necessary, surgical personnel remove an instrument from a manipulator arm and replace it with another.

One of the instruments 10 is a camera that captures images of the operative field in the body cavity. The camera may be moved by its corresponding robotic manipulator using input from a variety of types of input devices, including, without limitation, one of the handles 17, 18, additional controls on the console, a foot pedal, an eye tracker 21, voice controller, etc. The console may also include a display or monitor 23 configured to display the images captured by the camera, and for optionally displaying system information, patient information, etc. An auxiliary display 25, which may be a touch screen display, can further facilitate interactions with the system.

Sensors may optionally be used to determine the forces that are being applied to the patient by the robotic surgical tools during use. For example, a force/torque sensor on the surgical robotic manipulator may be used to determine the haptic information needed to provide force feedback to the surgeon at the console. U.S. Pat. No. 9,855,662, entitled Force Estimation for a Minimally Invasive Robotic Surgery System, describes a surgical robotic system in which sensors are used to determine the forces that are being applied to the patient by the robotic surgical tools during use. It describes the use of a 6 DOF force/torque sensor attached to a surgical robotic manipulator as a method for determining the haptic information needed to provide force feedback to the surgeon at the user interface. In the presently disclosed embodiments, a sensor of this type may optionally be positioned on or just proximal to the receiver 104.

As discussed, the input devices 17, 18 are configured to be manipulated by a user to generate signals that are processed by the system to generate instructions used to command motion of the manipulators in order to move the instruments in multiple degrees of freedom and to, as appropriate, control operation of electromechanical actuators/motors that drive instrument functions such as motion and/or actuation of the instrument end effectors. A control unit 30 is operationally connected to the robotic arms and to the user interface. The control unit receives user input from the input devices corresponding to the desired movement of the surgical instruments, and the robotic arms are caused to manipulate the surgical instruments accordingly.

This application comprises handles or tools used to give input to the system through the tracking of the handles or tools.

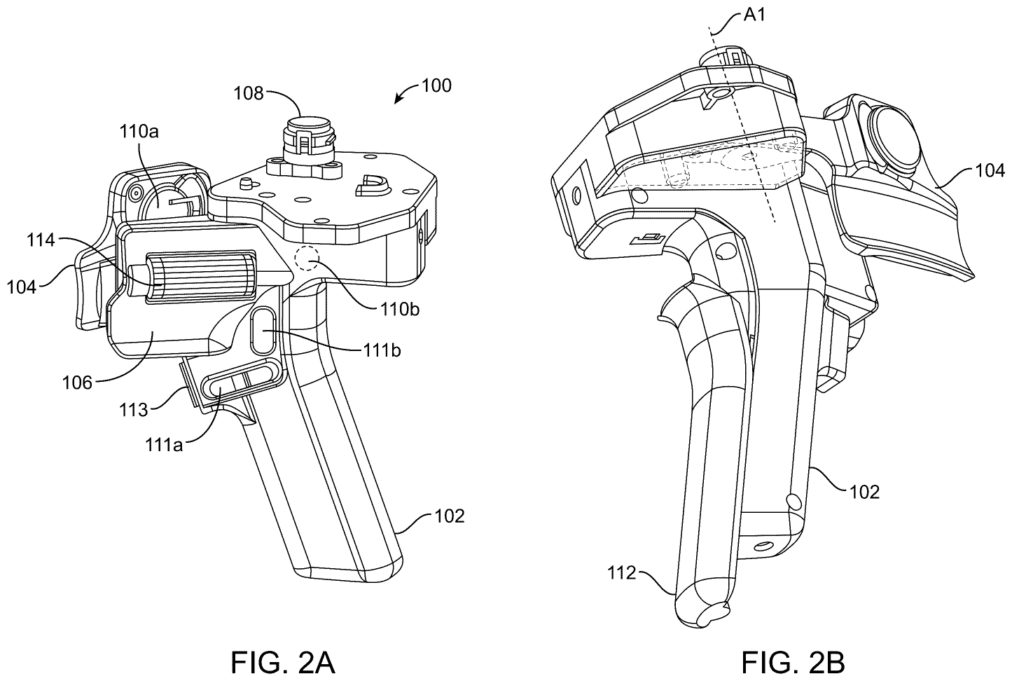

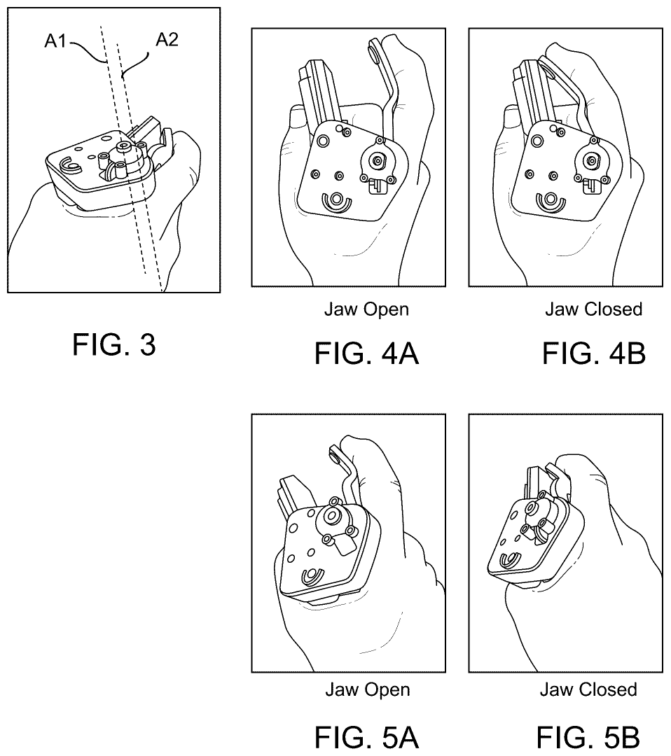

The handles shown herein are intended to be used as input devices to capture surgeon motion for a robotic surgical system. Referring to FIGS. 2A and 2B, an exemplary input tool/handle 100 preferably includes a fixed grip 102 and a moving lever 104. In preferred configurations, signals generated as a result of moving the lever 104 relative to the grip generate input for jaw open-close motion of the robotic surgical instrument. The grip 102 is preferably sufficiently long to seat against the user's palm in a stable fashion as depicted in FIG. 3. For example, it may be desirable that, when held as a pistol-grip, the user's pinky, ring finger and middle finger wrap at least partially around the grip 102, and the user's index finger seats against the lever 104. The user's thumb extends onto a distally-extending member or thumb rest 106 (which may be integrated with the grip). In preferred embodiments, the distally-extending member 106 does not pivot like the lever, but instead maintains a fixed position with respect to the grip 102.

Referring again to FIGS. 2A and 2B, the jaw lever positions, or changes in its position, are measured with an encoder 108 or other type of sensor. The encoder 108 may be a rotary encoder that uses optical, magnetic, inductive, capacitive, or resistive sensing methods. This encoder may output incremental relative position or absolute position data. The measured or detected jaw lever position may be translated into jaw closure or opening signals for the surgical instrument under control of the robotic system.

The jaw lever 104 may be lightly spring loaded so that it always applies pressure to the surgeon's index finger to keep the lever against the finger. Alternatively, a loop may be placed around the index finger to capture it and thus no spring would be necessary.

Referring to FIG. 3, the jaw lever has a primary rear axis A1 for open/close motion. This is the axis about which the jaw lever 104 pivots with respect to the grip 102. The axis's position is determined by the encoder 108 (FIG. 2A), which provides the position to the jaw closure algorithm of the robotic system, for instrument tip control. A unique aspect of the illustrated tool is the location of the lever pivot relative to the user's finger pivot. Rather than being centrally located like a pair of tweezers or other input handle types, the invention moves the pivot atop, or nearly adjacent to the user's index finger joint. This makes the motion more comfortable for the user, and the user's finger tip does not traverse down the length of the lever as the user moves the lever between opened and closed positions. As best illustrated in FIG. 3, the axis A1 is aligned, or nearly aligned with the open/close motion of the user's index finger, which occurs about axis A2. By contrast, in a more typical design, such as a tweezer-type configuration, the pivot axis of the lever is further offset from the user's index finger closure axis at the metacarpophalangeal joint. In preferred configurations, the offset between A1 and A2 is less than or equal to 1 cm, and in more preferred configurations it is less than or equal to 0.5 cm, or less than 0.25 cm.

FIGS. 4A-5B show the positions of the handles that result in jaw open (FIGS. 4A and 5A) and jaw close (FIGS. 4B and 5B) input to the robotic system.

Elements that provide haptic feedback such as vibration or sound may be incorporated into the device. These types of haptic actuators and/or motors can generate tactile feedback that communicates to the user certain events or conditions occurring at the surgical instrument. In the embodiment shown in FIG. 2A, a first haptic element 110a is positioned on the lever, allowing the user to feel haptic feedback at the user's index finger. This may communicate that instrument jaw forces at the surgical instrument have exceeded a predetermined level, or that the jaws have articulated to a fully closed position. FIG. 2A shows a second haptic element 110b positioned in the handle, allowing the user to feel haptic feedback on the palm or the base of the thumb.

As shown in FIG. 2B, some or part of the handle, such as the backstrap 112 that seats against the palm, may be replaceable with one or more alternative components/backstraps that change the shape or dimensions of the grip 102, allowing a surgeon to choose a grip that is ergonomic and comfortable in the surgeon's hand.

The input tool may include other features used to give varying types of input to the system, including a thumb button 111a and a slider switch 111b, both reachable by the thumb as shown. A trigger button 113 on the grip 102 may be reached by a user by removing his or her index finger from the lever 104 and moving it into position on the trigger button.

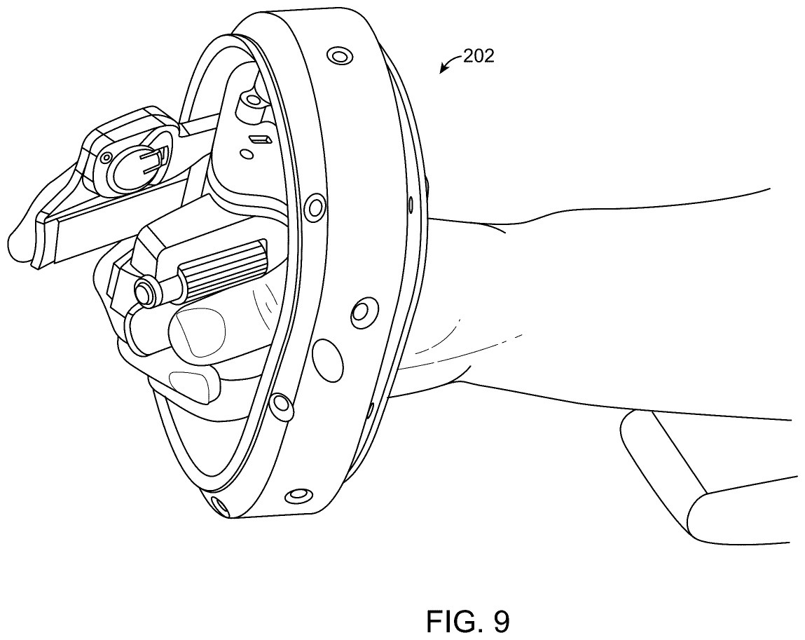

In some embodiments, a roll knob 114 may be incorporated into the handle as best shown in FIGS. 2A, 6 and 9, where the roll knob 114 is shown on the distally-extending member. As can be seen in FIG. 9, the roll knob may have an elongate cylindrical shape that a user can cause to roll about its longitudinal axis using the thumb. This roll knob may be used to provide rotation of the shaft of the surgical instrument about its longitudinal axis during teleoperation, or to provide axial roll of the tip of the surgical instrument relative to the instrument shaft. In other cases, the roll knob may be used to navigate through a menu or list of items on a GUI (graphical user interface). The roll knob may alternately be used to adjust a certain value or setting up and down. In some embodiments, the roll knob may be “clickable” as a button as well, for instance, to make a selection from such a list, or to select and confirm the value scrolled to via the roll knob. The functionality of the button may switch between multiple modes depending on the system state (e.g. during teleoperation, the roll knob may roll the shaft of the instrument, and when not in teleoperation and when navigating a list, the roll knob may be used to scroll through a list or adjust a value up and down).

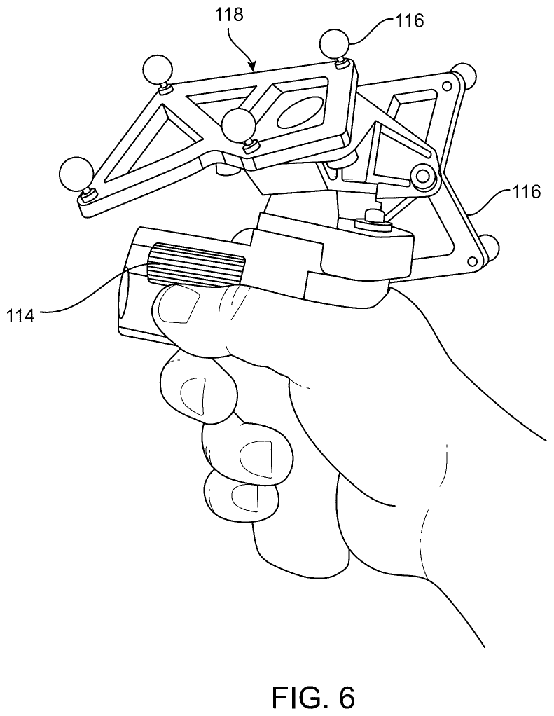

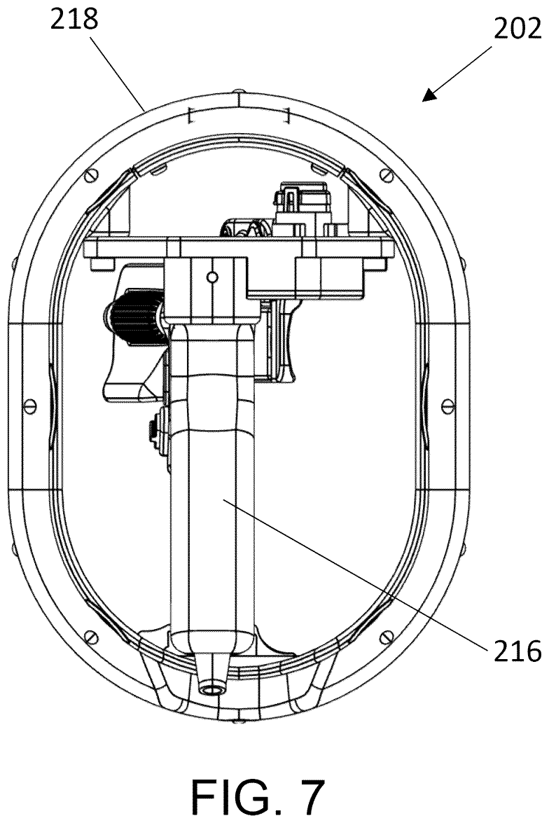

As discussed, in some uses of the disclosed handle, the handle's position and intended motion is captured via a tracking system which may use markers such as active (e.g. visible or infrared LEDs) or passive markers. These tracked items may be mounted onto the handles being tracked, or may be incorporated into the body of the handles themselves. The tracking markers are embedded into the handle or attached to the handle in a known configuration from which the 6 DoF position and velocity can be determined. The markers 116 may be positioned on a tracking frame 118 mounted to the handle. FIG. 6 shows one example of a tracking frame carrying tracking elements. FIGS. 7-9 show a second example of a tracking frame carrying tracking elements.

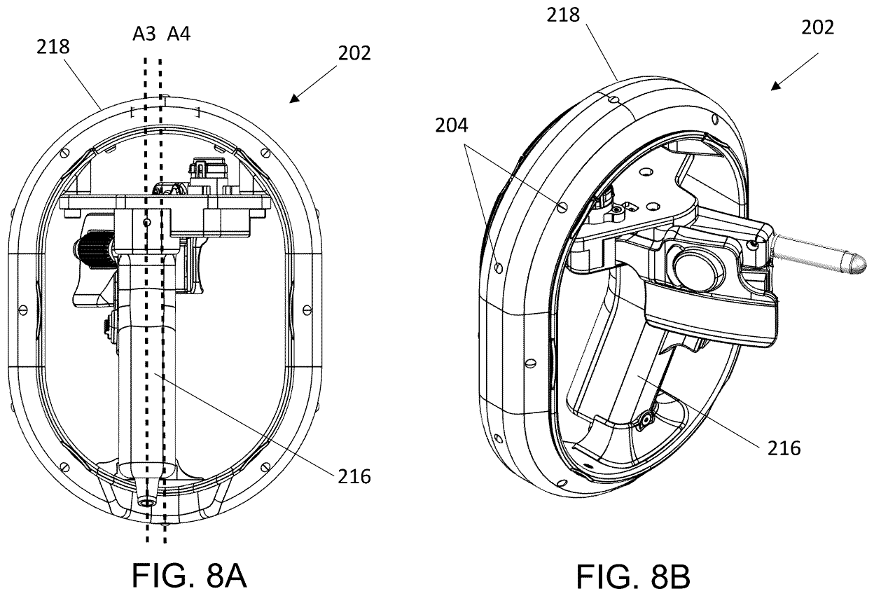

In the FIG. 7 embodiment, the frame is positioned encircling the grip. The illustrated frame has a stadium shape, but in alternative embodiments oval, elliptical, circular or other frame shapes may be used. As shown in the rear view of the handle shown in FIG. 8A, the longitudinal axis A3 of the grip extends parallel to the longitudinal axis A4 of the tracking frame. In an alternative embodiment using a circular frame is used, the longitudinal axis A1 of the grip might be parallel to the diameter of the circular frame.

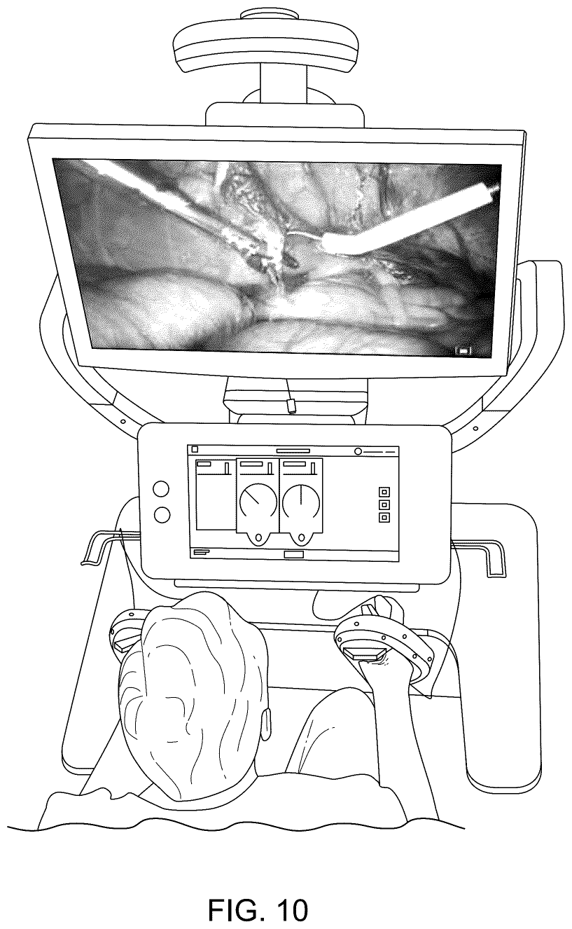

Tracking elements are disposed on the tracking frame as described in the referenced applications. As described, the described handles may be used with a variety of tracking modalities. In some examples, these tracking elements may be LEDS, such as IR LEDS, capable of being tracked using tracking cameras as described in the referenced application. The tracking elements are shown on multiple planes of the tracking geometry so as to provide rich tracking data to the handle tracking system. FIG. 10 shows a surgeon console using the tracking handles of FIGS. 8A-9 while observing real time images of the surgical site on a video display. Above the video display is a tracking which tracks the positions of the tracking elements. As described, the camera may be a more conventional tracking camera such as the Polaris Lyra, or it may use alternative cameras such event-based camera sensors.

In FIG. 10, a touch screen display is shown below the video display. The touch screen allows the user to provide input to the surgical system. In one embodiment of the input handles, a capacitive stylus may extend from the handle (e.g. on the distally-extending member as shown in FIG. 8A), allowing the user to use the capacitive stylus to touch the screen in order to give input or make selections when the handle is not actively controlling movement of a robotic manipulator or surgical instrument.

All prior patents and applications referenced herein, including for purposes of priority, are incorporated herein by reference.

1. A user input tool for providing provide input to a robotic surgical system for use in commanding motion of the robotic manipulator, the user input tool comprising:
a grip positionable in contact with a palm of a user, the user having an index finger with a metacarpophalangeal joint having a joint pivot axis;
a lever mounted to the grip and pivotable relative to the grip around a lever pivot axis;
wherein the lever pivot axis is offset from the joint pivot axis by 1 cm or less.

2. The user input tool of claim 1, wherein the lever pivot axis is offset from the joint pivot axis by 0.5 cm or less.

3. The user input tool of claim 1, wherein the lever pivot axis is offset from the joint pivot axis by 0.25 cm or less.

4. The user input tool of claim 1, wherein the lever pivot axis is parallel to the joint pivot axis.

5. The user input tool of claim 1, further including a thumb member positioned such that when the index finger is positioned against the lever, a thumb of the user is positioned against the thumb member.

6. The user input tool of claim 5, wherein the thumb member includes an elongate cylindrical member having an axis, the thumb member manually rotatable relative to its longitudinal axis.

7. The user input tool of claim 6, wherein the thumb member is in a fixed position relative to the grip.

8. The user input tool of claim 1, further including a touch screen stylus.

9. The user input tool of claim 1, further including a thumb member positioned such that when the index finger is positioned against the lever, a thumb of the user is positioned against the thumb member, wherein the touch screen stylus extends distally from the thumb member.

10. The user input tool of claim 1, further including a frame and a plurality of LEDs on the frame, the LEDs trackable by a user input system.

11. A method of operating a user input tool of a robotic surgical system, comprising:
grasping a user input tool such that a grip is positioned in contact with a palm of a hand having an index finger with a metacarpophalangeal joint having a joint pivot axis;
placing the index finger in contact with a lever mounted to the grip; and
using the index finger, pivoting the lever relative to the grip around a lever pivot axis, wherein the lever pivot axis is offset from the joint pivot axis by 1 cm or less.

12. The method of claim 11, wherein the lever pivot axis is offset from the joint pivot axis by 0.5 cm or less.

13. The method of claim 11, wherein the lever pivot axis is offset from the joint pivot axis by 0.25 cm or less.

14. The method of claim 11, wherein the lever pivot axis is parallel to the joint pivot axis.

15. The method of claim 11, further including positioning a thumb of the hand against the thumb member, such that the thumb is against the thumb member during pivoting of the lever relative to the grip.

16. The method of claim 15, wherein the thumb member includes an elongate cylindrical member having an axis, and wherein the method further includes using the thumb to axially rotate the thumb member relative to its longitudinal axis.

17. The method of claim 11, further including giving input to a touch screen using a touch screen stylus on the user input tool.

18. The method of claim 11, wherein the user input tool further includes user input tool a plurality of LEDs, and wherein the method further includes tracking the LEDs to determine a position and orientation of the user input tool, and robotically maneuvering a surgical instrument based on the position and orientation of the user input tool.
